(19) United States
(12) Patent Application Publication
(10) Pub. No.: US 2025/0114493 A1
(43) Pub. Date: Apr. 10, 2025

(54) HARD SURFACE RESIDUAL SANITIZATION OR DISINFECTION COATING AND METHODS FOR DOING THE SAME

(71) Applicant: The Clorox Company, Oakland, CA (US)

(72) Inventors: Mark Pszczolkowski, Pleasanton, CA (US); Kelly Dickson, Pleasanton, CA (US); Bradley Kirsch, Pleasanton, CA (US); Nikhil Dani, Pleasanton, CA (US)

(21) Appl. No.: 18/481,048

(22) Filed: Oct. 4, 2023

(51) Int. Cl. A61L 2/18; A61L 101/46

(52) U.S. Cl. CPC A61L 2/18; A61L 2101/46; A61L 2202/15

(57) ABSTRACT

System and methods for dispensing a residual surface sanitization or disinfection formulation on a surface. The system may include a formulation and a device. The device includes a sprayer configured to spray the formulation onto a surface as a coating. The spray has a Dv₅₀ particle size distribution of less than about 150 μm and the coating has a residual coating coverage efficiency of at least 30 cm²/mm². Additionally, the coating covers a majority of the surface and provides a durable coating on the surface for a time period therefore providing residual sanitization or disinfection to the surface over the time period.

### CROSS REFERENCE TO RELATED APPLICATIONS

N/A.

### BACKGROUND OF THE INVENTION

### 1. The Field of the Invention

The present invention relates to systems and methods for dispensing a residual surface sanitization or disinfection formula on a surface. The residual surface sanitization or disinfection formula may create a durable coating on the surface for a set time period that provides residual sanitization or disinfection on the surface over the time period.

### 2. Description of Related Art

Residual surface sanitization formulations and residual surface disinfection formulations are coatings that are applied onto a hard surface and allowed to dry. Once dried, the residual surface formulations inhibit viruses, bacteria and/or other microbes from growing on such surface. Such coatings are often a polymer-based coating which is applied with either a spray or wipe to the hard surface. While such residual surface formulations can in theory provide residual sanitization and/or disinfection of a surface, there is a continuing need for improved dispensing systems and methods that would more effectively result in a proper surface coating, particularly for hands-free application methods and systems, which do not require wiping of the surface, to achieve residual sanitization or disinfection.

For example, the coating needs to uniformly cover a majority (preferably significantly more than just a majority) of the surface while being resilient enough to stay set even when rubbed, abraded, or disturbed. Additionally, the coating must be thick enough to last for a suitable duration of time, such as 1 or more days, while also being resilient enough to handle disturbances of high traffic surfaces, such as kitchen counters, bathroom counters and the like.

If the coating is too thick, the formulation may be tacky to the touch and leave behind an undesirable and visible physical residue. If the coating is too thin, or coverage is too “spotty”, the formulation may not be able to provide proper residual sanitization or disinfection of the surface. If the coating fails to be applied uniformly over the surface, certain portions of the surface may not have sanitizing or disinfecting properties, while other areas may receive too much formulation, resulting in those areas being sticky, gloppy, and/or displaying unsightly residues. Additionally, it is desirable to apply the formulation in a spray-and-go fashion, or in other words, without any need for wiping or spreading the formulation after application.

### BRIEF SUMMARY

The present invention relates to systems and methods for dispensing a residual surface sanitization or disinfection formula on a surface. In more detail, a residual surface sanitization or disinfection formulation is a coating on a surface that provides sanitization or disinfection on the surface for a certain amount of time, after application. The present invention discloses methods for dispensing a residual surface sanitization or disinfection formula to maximize the residual coating coverage, particularly while using a hands-free, non-pressurized sprayer type device to deposit the formulation with a residual coating coverage efficiency (“RCCE”) above a given threshold value. For example, such a threshold value may be at least about 30 cm²/mm², where RCCE is defined as the observed surface area (in cm²) divided by the average hole surface area within such observed surface area, that is not covered by the spray (in mm²).

An exemplary embodiment is directed towards a system for dispensing a residual surface sanitization or disinfection formula on a surface, the system including a sanitization or disinfection formulation and a device. The device includes a sprayer configured to spray the formulation onto a surface as a coating and the spray has a Dv₅₀ particle size distribution of less than about 150 μm. The coating has a residual coating coverage efficiency of at least 30 cm²/mm² and covers a majority of the surface, providing a durable coating on the surface for a given time period and providing residual sanitization to the surface for such time period (e.g., such as 12 hours to about 1 month, or from 1 to 7 days).

Another embodiment is directed to a system for dispensing a residual surface sanitization or disinfection formula on a surface. The system includes a formulation that provides sanitization and/or disinfection to an applied surface against a target microbe and a device including a sprayer configured to spray the formulation onto the surface as a coating. The spray has a Dv₅₀ particle size distribution of no more than about 150 μm, a Dv₉₀ particle size distribution of no more than 500 μm, and a Dv₁₀ particle size distribution of no more than about 80 μm. The coating covers at least about 60% of the surface and provides a durable coating that remains present on the surface for a time period of from about 1 day to about 7 days, and the coating has a residual coating coverage efficiency of at least 30 cm²/mm².

Another embodiment is directed to a method for dispensing a residual surface sanitization or disinfection formula onto a surface. The method includes providing a device that includes a sprayer configured to spray a sanitization or disinfection formulation onto a surface as a coating, wherein the spray has an Dv₅₀ particle size distribution that is less than about 150 μm. The method also includes creating a coating on the surface, where the coating covers at least 50% of the surface, forms a durable coating that remains on the surface for at least about 12 hours, and has a residual coating coverage efficiency of at least 30 cm²/mm².

In an embodiment the time period is from about 12 hours to about 1 month.

In an embodiment the time period is from about 1 day to about 7 days.

In an embodiment the particle size distribution includes a Dv₁₀ particle size that is no more than about 80 μm, such as from about 50 μm to about 80 μm, and a Dv₉₀ particle size of less than 500 μm.

In an embodiment the coating is a polymer-based coating.

In an embodiment the coating is resilient to rubbing, abrasion, or other removal methods for the time period.

In an embodiment the formulation provides disinfection.

In an embodiment formulation provides sanitization.

In an embodiment the coating covers at least 60% of the surface.

In an embodiment the coating covers at least 70% of the surface.

In an embodiment the surface is a hard (e.g., non-porous) surface.

In an embodiment the coating is substantially transparent on the surface (e.g., substantially invisible to the unaided eye).

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIGS. 1A-1C show image results of coatings on a stainless steel surface using different exemplary and comparative devices.

FIGS. 2A-2C how image results of coatings on an ABS tile surface using different exemplary and comparative devices.

FIGS. 3A-3D show image results of coatings on an indicating paper surface using different exemplary and comparative devices.

### DETAILED DESCRIPTION OF THE PREFERRED EMBODIMENTS

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more surfactants.

Unless otherwise stated, all percentages, ratios, parts, and amounts used and described herein are by weight.

Numbers, percentages, ratios, or other values stated herein may include that value, and also other values that are about or approximately the stated value, as would be appreciated by one of ordinary skill in the art. As such, all values herein are understood to be modified by the term “about”. Such values thus include an amount or state close to the stated amount or state that still performs a desired function or achieves a desired result. A stated value should therefore be interpreted broadly enough to encompass values that are at least close enough to the stated value to perform a desired function or achieve a desired result, and/or values that round to the stated value. The stated values include at least the variation to be expected in a typical manufacturing or other process, and may include values that are within 10%, within 5%, within 1%, etc. of a stated value.

Some ranges may be disclosed herein. Additional ranges may be defined between any values disclosed herein as being exemplary of a particular parameter. All such ranges are contemplated and within the scope of the present disclosure.

In the application, effective amounts are generally those amounts listed as the ranges or levels of ingredients in the descriptions, which follow hereto. Unless otherwise stated, amounts listed in percentage (“%'s”) are in weight percent (based on 100% active) of any composition.

The formulation may provide sanitization, disinfection, or sterilization, other cleaning, or other treatment. As used herein, the term “sanitize” shall mean the reduction of “target” contaminants in the inanimate environment to levels considered safe according to public health ordinance, or that reduces a “target” bacterial population by significant numbers where public health requirements have not been established. By way of example, an at least 99% reduction in bacterial population within a 24-hour time period is deemed “significant.” Greater levels of reduction (e.g., 99.9%, 99.99%, etc.) are possible, as are faster treatment times (e.g., within 10 minutes, within 5 minutes, within 4 minutes, within 3 minutes, within 2 minutes, or within 1 minute), when sanitizing or disinfecting.

As used herein, the term “disinfect” shall mean the elimination of many or all “target” pathogenic microorganisms on surfaces with the exception of bacterial endospores.

As used herein, the term “sterilize” shall mean the complete elimination or destruction of all forms of “target” microbial life and which is authorized under the applicable regulatory laws to make legal claims as a “sterilant” or to have sterilizing properties or qualities.

Use of the term “residual” in combination with “sanitization”, “disinfection”, etc. means that the sanitization, disinfection, or similar effect continues even after application has occurred, for a given period of time (e.g., for at least 12 hours, at least 24 hours, at least 36 hours, at least 48 hours, at least 72 hours, etc.). Exemplary periods of time for the residual sanitization or disinfection effect may be for such time periods, up to 1 month, up to 2 weeks, up to 1 week, such as from 1-7 days.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Reference to ASTM or other standardized tests refers to the latest version of such standard, unless otherwise specified. Standards referenced herein are herein incorporated by reference in their entirety.

### II. Exemplary Dispensing Systems

In an aspect, the present invention is directed to dispensing of a residual surface sanitization or disinfection formula onto a surface. Disclosed embodiments dispense a residual surface formulation onto a surface in a substantially uniform way (e.g., more uniformly than can be achieved with an aerosol dispenser or a conventional trigger spray dispenser) using a hands-free, “non-pressurized” sprayer type device. Further, disclosed embodiments achieve coverage of the majority of the surface with the residual surface formulation as a coating where the coating is resilient enough to stay in place even when touched, rubbed, abraded, or similarly disturbed during normal use of such surface over a set time period. Additionally, the coating is sufficiently thick and provides sufficient coverage to provide sanitization and/or disinfection to the surface without being tacky to the touch or leaving an undesirable visible physical residue.

By way of example, disclosed embodiments include a sanitization or disinfection formulation and a device where the device includes a sprayer configured to spray the formulation onto the surface as a coating. The coating covers the majority of the surface to provide a durable coating on the surface over a given time period. The coating further provides residual sanitization and/or disinfection of the surface over at least the specified time period.

In an embodiment, the spray created by the sprayer of the device has a Dv₅₀ particle size distribution of less than about 200 μm, less than about 180 μm, less than about 150 μm, less than about 120 μm, such as about 100 μm, or such as about 50 μm to about 120 μm. In an embodiment, the spray has a Dv₉₀ particle size of less than about 700 μm, less than about 600 μm, less than about 500 μm, such as from about 200 μm to about 600 μm, or from about 300 μm to about 500 μm, or from about 400 μm to about 500 μm. In an embodiment, the spray has a Dv₁₀ particle size distribution of less than about 100 μm, less than about 90 μm, less than about 80 μm, such as from about 1 μm to about 100 μm, or 5 μm to about 80 μm, or from 20 μm to about 70 μm, such as about 40 μm, about 50 μm, about 60 μm, about 70 μm or about 80 μm. In a specific embodiment, the spray has a Dv₅₀ particle size distribution of no more than about 150 μm, a Dv₉₀ particle size of no more than about 500 μm, and a Dv₁₀ particle size of no more than about 80 μm.

The coating created by the spray from the device covers the majority of the surface to provide a durable coating. In an embodiment, the coating is a polymer-based coating. The coating is resilient against rubbing, abrasion, or other removal methods for a given time period. In some example embodiments, the surface is a hard surface (e.g., hard non-porous surface, such as a countertop, bathroom or kitchen tile, stainless steel surface, etc.). The coating is advantageously substantially transparent on the surface. In some example embodiments, the coating covers at least 50%, at least about 60%, at least about 70%, at least about 80%, at least about 90%, or above about 90% of the surface. In some embodiments, the coating is durable, providing residual sanitization or disinfection for from about 12 hours to about 2 months, from about 12 hours to about 1 month, from about 1 day to about 7 days, or from about 2 days to about 5 days.

The coating can be characterized by a residual coating coverage efficiency (“RCCE”) value. The residual coating coverage efficiency is defined as the observed surface area (in cm²) divided by the average hole surface area size (in mm²) not covered by the coating. As a result, the residual coating coverage efficiency is measured in cm²/mm². The residual coating coverage efficiency as described herein is measured over a single pass of spray on a given surface without additional interference, such as wiping, spreading, or other changes to the initially sprayed surface, which is allowed to dry, and then photographed and measured.

To effectively cover the surface, the coating should have a residual coating coverage efficiency above a given threshold. In some embodiments, the threshold may be above about 30 cm²/mm², above about 40 cm²/mm², or above about 50 cm²/mm². By way of example, an exemplary disclosed embodiment using a FLAIROSOL spray dispenser has a residual coating coverage efficiency of about 62 cm²/mm². Such value is significantly higher than the comparative aerosol sprayer, and trigger sprayer, which only achieved RCCE values of 18 and 25, respectively.

Disclosed embodiments include a formulation that provides either sanitization and/or disinfection to an applied surface. In some instances, the formulation provides sanitization and/or disinfection against a target microbe, such as Staphylococcus aureus. Other target microbes will be apparent to those of skill in the art, e.g., in light of relevant EPA or other regulatory testing protocols. In some exemplary embodiments, the formulation may provide only sanitization, only disinfection, or both sanitization and disinfection. Non-limiting examples of formulations that may be suitable for use include those available under the tradename MICROBAN. Non limiting examples are disclosed in U.S. Pat. No. 11,134,678 to Lan et al.; U.S. Pat. No. 11,134,674 to Lan et al.; U.S. Pat. No. 11,033,023 to Lan et al.; U.S. Pat. No. 11,026,418 to Lan et al.; U.S. Pat. No. 10,925,281 to Lan et al.; U.S. Pat. No. 10,842,147 to Lan et al.; and U.S. Pat. No. 10,834,922 to Lan et al, each of which is herein incorporated by reference in its entirety. In an embodiment, the formulation may have a viscosity of less than 1000 cps, such as from about 1-1000 cps, about 1-500 cps, or about 100-500 cps.

Disclosed embodiments also include a device including a sprayer configured to spray the formulation onto the surface as a coating, at the high RCCE values noted. In particular, in an embodiment, the device is a hands-free (i.e., hand wiping of the formulation is not required), non-pressurized, continuous sprayer type of device, such as those available under the tradename FLAIROSOL. Non-limiting examples of devices are disclosed in U.S. Pat. No. 8,905,271 to Maas et al.; U.S. Pat. No. 11,660,624 to Maas et al.; U.S. Pat. No. 10,456,798 to Maas et al.; U.S. Pat. No. 10,562,053 to Maas et al.; and U.S. Pat. No. 9,714,133 to Maas et al., each of which is herein incorporated by reference in its entirety. Such continuous spray devices differ from typical trigger spray devices in that actuation of the trigger builds up pressure within a pressure reservoir of the device, and a substantially continuous pressure is applied to the formulation as it is dispensed, rather than direct application of the pressure to the formulation, as occurs within a conventional trigger spray device, which causes wild fluctuations in pressure applied to the formulation, during dispensing. Such devices also differ from conventional pressurized aerosol dispenser devices, that rely on a pressurized propellant packaged within the aerosol dispensing device, which is used to deliver the formulation from the aerosol dispenser. Such aerosol dispensers are notorious for delivering wildly different particle size distribution characteristics depending on whether the aerosol can is nearly full (i.e., top of the can) versus nearly empty (i.e., bottom of the can). In addition, the compressed gas propellants employed, particularly those that provide the best dispensing characteristics, are environmental pollutants, and therefore their use is undesirable.

By way of example, in an embodiment, using a dispenser such as a FLAIROSOL dispenser, a relatively low flow rate (e.g., 1-2 cm³ per actuation) may be provided, without dribbling of the formulation at the nozzle. A pressure check valve may be provided, to minimize and/or prevent any such dribbling. Advantageously, the dispenser may be refillable, and reusable, while providing the described particle size distribution and RCCE characteristics.

Alternative devices for spraying are also possible, e.g., where such sprayers can meet the variously described characteristics relative to RCCE and particle size distribution. In an embodiment, application through an electrostatic sprayer may meet such requirements.

Additionally, the above system may be incorporated into a method of use in some embodiments. For example, a method for dispensing a residual surface sanitization or disinfection formula onto a surface to be treated includes providing a device that includes a sprayer configured to spray a formulation onto a surface. The device may be any of the example devices described herein, or other appropriate devices configured to provide the described particle size distribution and RCCE characteristics. The formulation may be any of the example formulations listed above or other appropriate formulations configured for residual surface sanitization and/or disinfection. In example embodiments the spray has a Dv₅₀ particle size distribution that is less than about 150 μm.

The method further includes creating a coating on the surface. In an example embodiment, the coating covers at least 50% of the surface, forms a durable coating that remains on the surface for at least about 12 hours, and has a residual coating coverage efficiency of at least 30 cm²/mm². In some embodiments, the coating is a polymer-based coating. Exemplary polymers that may be used include polyvinylpyrrolidone and quat containing polymers, such as described in the formulation patents already incorporated by reference. Optionally, the formulation may provide only disinfection, only sanitization, or a combination of both disinfection and sanitization.

Advantageously, the disclosed systems and methods for dispensing a residual sanitization or disinfection formula onto a surface may provide a consistent, substantially uniform application or dosing of the chemistry found in the formulation for optimal usage as a residual surface disinfection and/or sanitization formula. Additionally, disclosed embodiments may provide an appropriately thick coating to provide disinfection and/or sanitization over a time period without creating a tacky to the touch coating or leaving undesirable physical residue on the surface. For example, the coating thickness (e.g., average coating thickness) may be no more than 500 μm, no more than 400 μm, no more than 300 μm, no more than 200 μm, or no more than 100 μm, such as from 50 μm to 200 μm, or 50 μm to 150 μm.

The dispensing method may provide a durable coating that is resilient enough even when rubbed, abraded, or disturbed over the set time period. Other advantages include maximizing residual coating coverage by using a residual coating coverage efficiency rating to determine the efficiency of the deposited formula on the surface.

### III. Examples and Data

Turning now to the Figures, the Figures provide various experimental data for formulas dispensed using different devices on different types of surfaces. From the experimental images and data, residual coating coverage efficiency values were calculated by taking the observed surface area (cm²) divided by the average hole surface area size not covered by the formulation (mm²). Each experiment was conducted by a single pass of spray over a given surface without additional spreading, wiping, or other distribution methods.

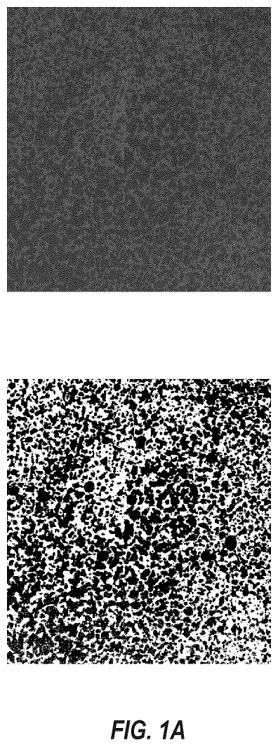

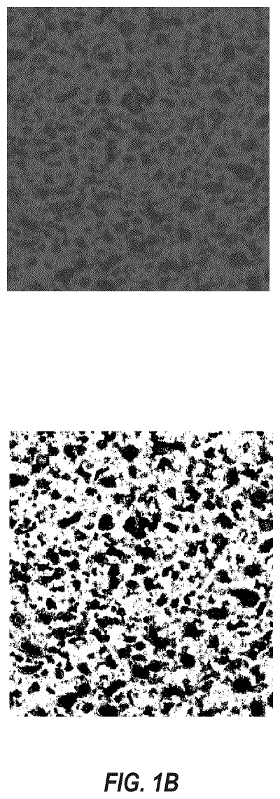

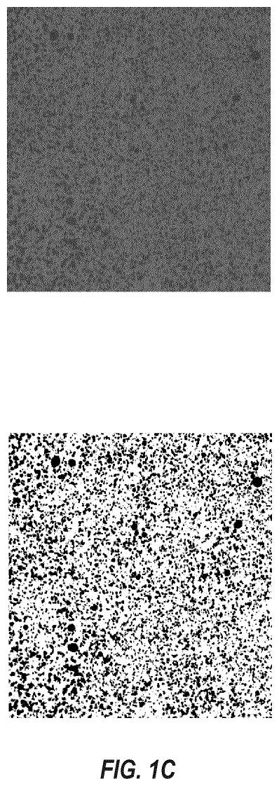

FIGS. 1A through 1C illustrate images of a MICROBAN formulation applied onto a stainless steel tile using different dispensing devices. The top and bottom images for each Figure are similar, with each bottom image including greater contrast.

FIG. 1A illustrates experimental images of MICROBAN on a stainless steel tile applied using a conventional hand-held trigger spray device. As seen, traditional trigger sprayer devices create a relatively wide particular size distribution dominated by relatively large particles, creating some areas with large amounts of formula (which will result in a sticky or tacky residue) and other areas with sparse amounts of formula (where no residual sanitization and/or disinfection will be achieved). In other words, the traditional trigger sprayers fail to create a consistent, substantially uniform spread of formula on the surface, with the needed particle size distribution characteristics that would be needed for residual sanitization or disinfection of such surface. Table 1 below shows the coverage characteristics associated with this image. One particular disadvantage of the traditional trigger sprayer is once this formulation dries on the surface, particular areas with large amounts of product will become sticky or tacky due to an excess amount of formulation and other areas with sparse amounts of product may not exhibit appropriate sanitization and/or disinfection properties.

FIG. 1B illustrates experimental images of MICROBAN on a stainless steel tile applied using a conventional aerosol can sprayer device. As seen in the Figures, the particle size distribution of the formula on the surface is even worse that that associated with the conventional trigger sprayer of FIG. 1A, showing a coverage pattern that is extremely inconsistent, with very large particle sizes scattered among some smaller particle size coverage areas. The nature of the pressurized propellant used in an aerosol sprayer device leads to inconsistencies in formula distribution when dispensing. Similarly, to the trigger sprayer device, the aerosol sprayer device will result in sticky or tacky areas on the surface once the formula dries and other areas on the surface where sanitization and/or disinfection properties may not be achieved due to the sparse formula distribution within some areas. A further disadvantage with aerosol sprayer devices is the inconsistencies with application over the lifetime of the aerosol can. For example, the aerosol sprayer device may distribute more formula at the beginning of the use of the device (e.g., when the aerosol can is nearly full) compared to the end of the use of the device (e.g., when the aerosol can is nearly empty). This results in extreme variability and inconsistency of the product over the lifetime of the aerosol sprayer device. In addition, there is also inconsistency with such spray devices from one production lot to the next.

FIG. 1C illustrates experimental images of MICROBAN applied on a stainless steel tile using a FLAIROSOL reservoir pump continuous spray device. As seen, the particle size distribution of the formula on the surface is significantly more uniform and consistent, with significantly smaller overall average particle size, and tighter particle size distribution. As shown in this example, FLAIROSOL is one example device which may be used in the disclosed embodiments to provide an effective coating on the surface that is both substantially consistent and uniform.

Quantitative data relating to FIGS. 1A through 1C will now be discussed, in particular, the residual coating coverage efficiency value for dispensing the formula on the stainless steel tile for each dispensing device type. As mentioned above, the residual coating coverage efficiency is calculated by dividing the observed surface area (23.02 cm² for each example) by the average hole surface area size not covered by the formula. Table 1 also shows the percentage of the observed area that is actually covered by the applied composition, for each dispensing device. The results are summarized in Table 1 below.

TABLE 1

| | | | | Residual |
|---|---|---|---|---|
| | | Average Hole | | Coating |
| | Observed | Surface Area Size | | Coverage |
| | Surface | Not Covered by | Coverage | Efficiency |
| Dispenser | area (cm²) | Formula (mm²) | (%) | (cm²/mm²) |
| Trigger | 23.02 | 0.91 | 44.2 | 25.3 |
| Sprayer | | | | |
| Aerosol | 23.02 | 1.28 | 62.8 | 18.0 |
| Sprayer | | | | |
| FLAIROSOL | 23.02 | 0.37 | 73.0 | 62.2 |
| Sprayer | | | | |

As shown in FIGS. 1A through 1C and Table 1, the aerosol sprayer performed the worst, followed by the trigger sprayer and the FLAIROSOL sprayer performed the best. In this example experiment, only the FLAIROSOL dispenser meets the threshold value for residual coating coverage efficiency of at least 30 cm²/mm². Such characteristics correlate with the ability to create a substantially uniform, consistent coating without being sticky or tacky, and without leaving an unsightly visible residue while still providing effective sanitization and/or disinfection to the stainless steel surface for a given period of time, under normal usage conditions (e.g., while being touched, etc. during normal use).

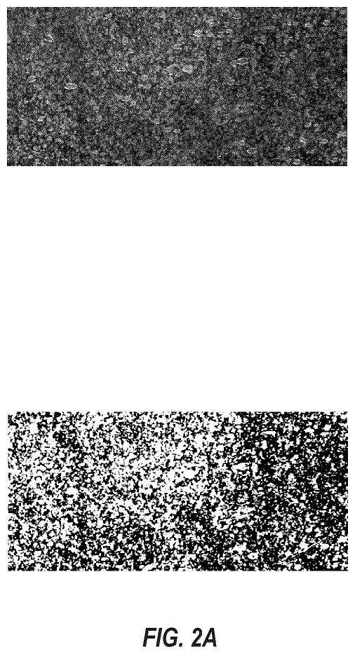

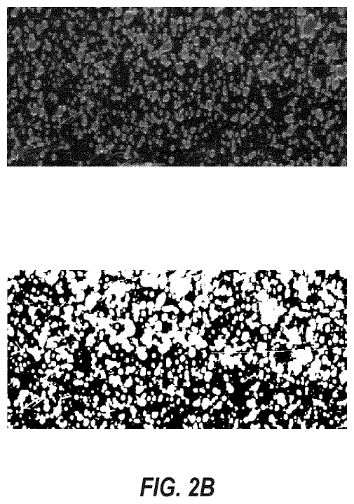

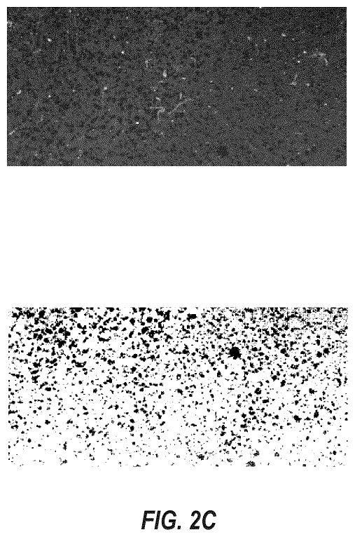

Turning now to FIGS. 2A through 2C, the Figures illustrate images of MICROBAN product as applied onto an acrylonitrile butadiene styrene (ABS) tile using different exemplary and comparative devices. The top and bottom images for each Figure are similar, with each bottom image including greater contrast.

FIG. 2A illustrates experimental images of MICROBAN applied on an ABS tile using a trigger spray device. As seen, traditional trigger sprayer devices result in a wide particular size distribution, with large particles, creating areas with large amounts of formula and other areas with sparse amounts of formula. In other words, the traditional trigger sprayers fail to create a substantially consistent, uniform spread of formula on the surface. One particular disadvantage of the traditional trigger sprayer is once this formulation dries on the surface, particular areas with large amounts of product (due to the dominant large particle sizes) will become tacky due to an excess amount of formulation and other areas with sparse amounts of product may not exhibit appropriate sanitization and/or disinfection properties.

FIG. 2B illustrates experimental images of MICROBAN applied on an ABS tile using an aerosol spray device. As seen in the Figures, the particle size distribution of the formula on the surface is extremely inconsistent resulting in the aerosol sprayer device performing even worse than the trigger sprayer device. Similarly, to the trigger sprayer device, the aerosol sprayer device will result in sticky or tacky areas on the surface once the formula dries and other areas on the surface where sanitization and/or disinfection properties may not occur due to the sparse formula distribution. A further disadvantage with aerosol sprayer devices is the inconsistencies in application over the lifetime of the particular aerosol can, as described above.

FIG. 2C illustrates experimental images of MICROBAN applied on an ABS tile using a FLAIROSOL reservoir pump continuous spray device. As seen, the particle size distribution of the formula on the surface is both substantially uniform and consistent, dominated by smaller particle sizes by comparison to FIGS. 2A-2B. As shown in this example, FLAIROSOL is one example device which may be used in the disclosed embodiments to provide an effective coating on the surface that is both substantially consistent and uniform.

The results are summarized in Table 2 below.

TABLE 2

| Dispenser | Observed Surface area (cm²) | Coverage (%) |
|---|---|---|
| Trigger Sprayer | 12.74 | 48.0 |
| Aerosol Sprayer | 12.74 | 47.7 |
| FLAIROSOL Sprayer | 12.74 | 83.0 |

As shown in FIGS. 2A through 2C and Table 2, the aerosol sprayer performed the worst, followed by the trigger sprayer and the FLAIROSOL sprayer performed the best. In this example experiment, only the FLAIROSOL dispenser meets the threshold value for residual coating coverage efficiency of at least 30 cm²/mm². Such characteristics correlate with the ability to create a substantially uniform, consistent coating without being sticky or tacky, and without leaving an unsightly visible residue while still providing effective sanitization and/or disinfection to the ABS surface for a given period of time, under normal usage conditions (e.g., while being touched, etc. during normal use).

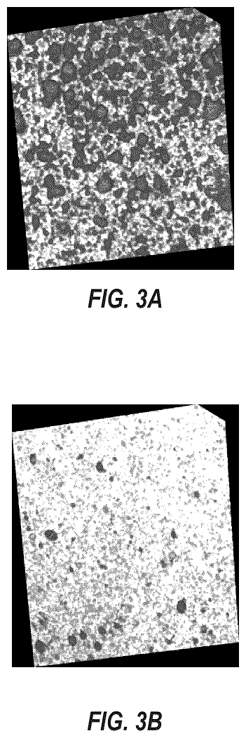

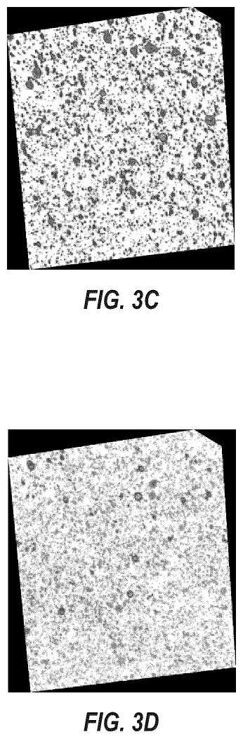

FIGS. 3A through 3D illustrate images of MICROBAN product applied onto indicating paper using different devices.

FIGS. 3A and 3B illustrate experimental images of MICROBAN applied onto indicating paper using a trigger sprayer device. In more detail, FIG. 3A illustrates the spray pattern achieved at the center of the spray while FIG. 3B illustrates the spray pattern achieved at the edge of the spray. As seen, traditional trigger sprayer devices result in a very wide particular size distribution creating areas with large amounts of formula (e.g., large particles, and high coverage near the center) and other areas with sparse amounts of formula (significantly smaller particles, at the edge of the spray pattern). In other words, the traditional trigger sprayers fail to create a consistent, substantially uniform spread of formula on the surface. One particular disadvantage of the traditional trigger sprayer is once this formulation dries on the surface, particular areas with large amounts of product (e.g., near the center of the spray pattern) will become sticky or tacky due to an excess amount of formulation and other areas (near the peripheral edges of the spray pattern) with sparse amounts of product may not exhibit appropriate sanitization and/or disinfection properties.

FIG. 3C illustrates experimental images of MICROBAN applied onto indicating paper using an aerosol sprayer device. While an aerosol spray device may not have the same variability from center to edge as exhibited with a trigger sprayer. As seen in FIG. 3C, the particle size distribution of the formula on the surface is extremely broad and inconsistent with very large particles, and quite small particles, but dominated by the relatively larger particles, within the spray pattern. Similarly, to the trigger sprayer device, the aerosol sprayer device will result in tacky areas on the surface once the formula dries and other areas on the surface where sanitization and/or disinfection properties may not occur due to the sparse formula distribution.

FIG. 3D illustrates experimental images of MICROBAN applied onto indicating paper using a FLAIROSOL spray device. As seen, the particle size distribution of the formula on the surface is significantly more uniform and consistent. As shown in this example, FLAIROSOL is one example device which may be used in disclosed embodiments to provide an effective coating on the surface that is both consistent and substantially uniform.

Lastly, exemplary particle size distribution data was determined for the trigger sprayer, aerosol sprayer, and the FLAIROSOL sprayer, all applying the same MICROBAN formulation. The Dv₁₀, Dv₅₀, Dv₉₀, D3,2, and D4,3 particle size characteristics were determined in μm for each sprayer type. The particle size data was obtained by averaging the data over a spray timespan of at least 5 seconds. The aerosol and FLAIROSOL measurements were taken with the dispenser nozzle 6 inches away from the surface, while the trigger sprayer measurements were taken with the dispenser nozzle 12 inches from the surface. The trigger sprayer moved further from the application surface, due to the spray pattern provided by the trigger sprayer being more focused at the same dispensing distance as compared to the other sprayer types. The experimental results on particle size distribution are summarized in Table 3 below.

TABLE 3

| | Dv₁₀ | Dv₅₀ | Dv₉₀ | D3,2 | D4,3 | |
|---|---|---|---|---|---|---|
| | (μm) | (μm) | (μm) | (μm) | (μm) | Span |
| FLAIROSOL | 53.58 | 103.3 | 451.8 | 90.58 | 225.8 | 3.855 |
| Aerosol | 88.75 | 170.2 | 704.2 | 150.1 | 289.2 | 3.617 |
| Bottom of Can | | | | | | |
| Aerosol Top | 70.97 | 126.8 | 230.6 | 111.3 | 155.9 | 1.259 |
| of Can Test #1 | | | | | | |
| Aerosol Top | 100 | 230 | 500 | — | — | — |
| of Can Test #2 | | | | | | |
| Trigger | 66.69 | 140.4 | 698.9 | 119.8 | 286.2 | 4.503 |
| Sprayer | | | | | | |

As shown in Table 3, the FLAIROSOL device has the lowest Dv₅₀ (median particle size) compared to the other device types. Additionally, the FLAIROSOL also has a significantly smaller Dv₁₀ particle size as compared to the other device types. The aerosol sprayer type shows inconsistencies throughout lifetime of a given can, as seen by the differences between the data provided for the bottom of the can versus the top of the can. In addition, the 2^{nd} tested aerosol can illustrates how there is additional variability from one production lot to the next with the aerosol sprayer device. Lastly, the trigger sprayer device shows larger particle sizes than the FLAIROSOL at every measurement point (Dv₉₀, Dv₅₀, Dv₁₀, etc.), making it a less effective dispensing device.

Without departing from the spirit and scope of the invention, one of ordinary skill can make various changes and modifications to the invention to adapt it to various usages and conditions. As such, these changes and modifications are properly and intended to be, within the full range of equivalence of the following claims.

1. A system for dispensing a residual surface sanitization or disinfection formula on a surface, the system comprising:
a sanitization or disinfection formulation; and
a device including a sprayer configured to spray the formulation onto a surface as a coating, wherein:
the spray has a particle size distribution including a Dv₅₀ particle size distribution of less than about 150 μm;
the coating having a residual coating coverage efficiency of at least about 30 cm²/mm²; and
the coating covers a majority of the surface and provides a durable coating on the surface for a time period so as to provide residual sanitization and/or disinfection to such surface for such time period.

2. The system of claim 1, wherein the time period is from about 12 hours to about 1 month.

3. The system of claim 2, wherein the time period is from about 1 day to about 7 days.

4. The system of claim 1, wherein the particle size distribution includes a Dv₁₀ particle size from about 50 μm to about 80 μm, and a Dv₉₀ particle size of less than about 500 μm.

5. The system of claim 1, wherein the coating is a polymer-based coating.

6. The system of claim 1, wherein the coating is resilient to rubbing, abrasion, or other removal methods for the time period.

7. The system of claim 1, wherein the formulation provides disinfection.

8. The system of claim 1, wherein the formulation provides sanitization.

9. The system of claim 1, wherein the coating covers at least about 60% of the surface.

10. The system of claim 9, wherein the coating covers at least about 70% of the surface.

11. The system of claim 1, wherein the surface is a hard surface.

12. The system of claim 1, wherein the coating is substantially transparent on the surface.

13. A system for dispensing a residual surface sanitization or disinfection formula on a surface, the system comprising:
a formulation, wherein the formulation provides sanitization and/or disinfection to an applied surface against a target microbe; and
a device including a sprayer configured to spray the formulation onto the surface as a coating, wherein:
the spray has a Dv₅₀ particle size distribution of no more than about 150 μm, a Dv₉₀ particle size distribution of no more than about 500 μm, and a Dv₁₀ particle size distribution of no more than about 80 μm;
the coating covers at least about 60% of the surface and provides a durable coating that remains present on the surface for a time period of from about 1 day to about 7 days, and
the coating having a residual coating coverage efficiency of at least about 30 cm²/mm².

14. The system of claim 13, wherein the coating is a polymer-based coating.

15. The system of claim 13, wherein the surface is a hard surface.

16. The system of claim 13, wherein the coating is substantially transparent on the surface.

17. A method for dispensing a residual surface sanitization or disinfection formula onto a surface, the method comprising:
providing a device that includes a sprayer configured to spray a formulation onto a surface as a coating, wherein the spray has an Dv₅₀ particle size distribution that is less than about 150 μm; and
creating a coating on the surface, wherein the coating covers at least about 50% of the surface, forms a durable coating that remains on the surface for at least about 12 hours, and has a residual coating coverage efficiency of at least about 30 cm²/mm².

18. The method of claim 17, wherein the coating is a polymer-based coating.

19. The method of claim 17, wherein the formulation provides disinfection.

20. The method of claim 17, wherein the formulation provides sanitization.
